(19) United States
(12) Patent Application Publication
(10) Pub. No.: US 2008/0102050 A1
(43) Pub. Date: May 1, 2008

(54) WATER-BASED SKIN CARE COMPOSITION WITH POLYALKYLSILSESQUIOXANE POWDER EMULSION AND METHOD FOR MAKING THE SAME

(76) Inventors: Mingxla Li, Puxi Shanghai (CN); Tian Dong Li, Jurong East (SG)

Correspondence Address: GENERAL ELECTRIC CO.;GLOBAL PATENT OPERATION, 187 Danbury Road, Suite 204, Wilton, CT, 06897-4122, US

(21) Appl. No.: 11/553,284

(22) Filed: Oct. 26, 2006

(51) Int. Cl. A61K 8/89

(52) U.S. Cl. 424/70.12

(57) ABSTRACT

A process for making a water-based skin care composition including blending a polyalkylsilsesquioxane powder emulsion and water. A skin care composition made by the process is also presented, which is easy to prepare and do not require additional heating or further emulsification of the skin care formulation.

### FIELD OF THE INVENTION

This invention relates to skin care compositions. More specifically, this invention relates to water-based skin care compositions comprising a polyalkylsilsesquioxane powder emulsion.

### BACKGROUND OF THE INVENTION

Water-based skin care products, such as facial masks, wipes, rinse-off skin care products and oil-in-water creams and lotions are popular cosmetic products that have useful applications for adults and children. Water-based skin care compositions are applied to the skin for cleansing or skin conditioning and are appealing to consumers for their ease of use. Despite their benefits, these products do not deliver a long-lasting smooth and soft feeling to the skin. Additionally, many consumers would like to have a water-based product that both conditions the skin and helps hide wrinkles.

U.S. Pat. No. 4,892,726 discloses oil-based cosmetic compositions containing polymethylsilsesquioxane powder having a smooth application. However, polyalkylsilsesquioxane powder is not compatible with aqueous compositions or with the aqueous phase of water-based compositions.

U.S. Pat. No. 5,643,555 discloses polymethylsilsesquioxane powder in a water-in-oil emulsion that requires a complex emulsification process.

JP Laid-open application No. 5-310533 discloses hair cosmetic compositions containing polymethylsilsesquioxane powder and water that requires heating to disperse the powder within the product. Heating a skin care cosmetic composition may prevent the use of some ingredients that are heat-sensitive within the formulation and the process of heating or further emulsifying the skin care composition is time-consuming, difficult to control and requires expensive equipment for preparation.

What is needed is a water-based skin care composition that provides improved conditioning and a long lasting smooth and soft feeling to the skin with improved hiding of wrinkles and a method of making a water-based skin care composition with simple blending of the formulation and without having to additionally heat or further emulsify the water-based skin care formulation.

### SUMMARY OF THE INVENTION

In one embodiment, a process for making a water-based skin care composition comprises blending a polyalkylsilsesquioxane powder emulsion and water.

In another embodiment, a water-based skin care cosmetic composition comprises a polyalkylsilsesquioxane powder emulsion having an average particle diameter in the range of about 0.05 μm to about 100 μm, an emulsifier and water, wherein said emulsifier is less than about 15 percent by weight and the water is greater than about 6 percent by weight, wherein said percent by weight is based on the total weight of the composition.

In another embodiment, an emulsion comprises polyalkylsilsesquioxane powder having an average particle diameter in the range of about 0.05 μm to about 100 μm, a silicone, an emulsifier and water, wherein said emulsifier is less than about 15 percent by weight and the water is greater than about 6 percent by weight, wherein the percent by weight is based on the total weight of the emulsion.

The various embodiments provide skin conditioning properties, such as excellent long lasting smooth and softness touch, an emollient feel, enhanced blurring of fine lines and hiding of wrinkles, favorable natural color and good compatibility and dispersion in water-based skin care compositions. The cosmetic compositions are easy to prepare and do not require additional heating or further emulsification of the skin care formulation.

### DETAILED DESCRIPTION OF THE INVENTION

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

The modifier “about” used in connection with a quantity is inclusive of the stated value and has the meaning dictated by the context (e.g., includes the tolerance ranges associated with measurement of the particular quantity).

“Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present.

In one embodiment, a process for making a water-based skin care composition comprises blending a polyalkylsilsesquioxane powder emulsion and water.

A water-based skin care composition is a composition, which contains water as a solvent or as a dispersing medium for the ingredients in the skin care formulation. The composition may be an oil-in-water emulsion or an aqueous solution.

The polyalkylsilsesquioxane powder emulsion is obtained by dispersing polyalkylsilsesquioxane powder in a water phase with agitation. Emulsifiers may be added to aid in the emulsion preparation. The mixture of the polyalkylsilsesquioxane powder, water and optional emulsifiers may be heated to aid in the emulsion preparation. The mixture may be heated to melt and may be heated at a temperature greater than or equal to about 55° C. including a range of from about 55° C. to about 90° C.

The emulsion is preformed before it is added to the skin care formulation. The preformed emulsion allows dispersion of the polyalkylsilsesquioxane powder in the water-based composition with simple blending and without additional heating or further emulsification. Heating limits the kinds of ingredients that may be added to the skin care formulation, as many of the ingredients may be heat-sensitive and degrade with heating. In addition, heating and emulsification processes may be difficult to control, are time-consuming and require expensive equipment for preparation. The use of a preformed emulsion provides flexibility in preparing different types of skin care compositions, as only minimal equipment change is needed to make a different type of skin care composition.

Polyalkylsilsesquioxane is represented by the average unit formula of R¹SiO_{1.5}, where R¹ is a substituted or unsubstituted monovalent hydrocarbon group, including a C₁-C₃₀ alkyl group, a C₂-C₃₀ alkenyl group, a C₆-C₃₀ aryl group, a C₃-C₃₀ cycloalkyl group and groups formed by replacing one or more of the carbon-bonded hydrogen atoms in those groups with halogen atom, epoxy, methacyloxy, carboxyl, carboxylate radical, sulfhydryl, polyoxyalkylene, mercapto, hydroxyl, cyano group, amino group or other substituent, such as chloromethyl, 3,3,3-trifluoropropyl, cyanomethyl, γ-aminopropyl and N-(β-aminoethyl)-γ-aminopropyl. Examples of alkyl groups are methyl, ethyl, propyl, isopropyl, butyl, tert-butyl, octyl, lauryl and stearyl groups. Examples of alkenyl groups are vinyl, allyl and propenyl. Examples of aryl groups are phenyl, tolyl, naphthyl, benzyl, phenylethyl groups, 2-phenylpropyl and xylyl. Examples of cycloalkyl groups are cyclopentyl, cyclohexyl and cyclooctyl.

In one embodiment, the polyalkylsilsesquioxane is polymethylsilsesquioxane, polyethylsilsesquioxane or polyphenylsilsesquioxane.

Polyalkylsilsesquioxane powder can be synthesized by any conventional method. It may be prepared by condensing a trifunctional hydrolysable silane, such as an alkyltrichlorosilane or a trialkoxysilane. An alkyltrichlorosilane, such as methyltrichlorosilane, is hydrolyzed by spraying it or dropwise adding it to large amounts of water to cause hydrolysis and condensation. A trialkoxysilane may be hydrolyzed and condensed in an aqueous solution containing alkali metal carbonates or alkaline earth metal hydroxides or in aqueous solutions of ammonia or amine.

The trialkoxysilane is represented by the general formula

R¹Si(OR²)₃

in which R¹ is a substituted or unsubstituted monovalent hydrocarbon group and R² is a hydrogen atom or a monovalent hydrocarbon group having from 1 to 6 carbon atoms, or a partial hydrolysis-condensation product thereof.

The R¹ group is a substituted or unsubstituted monovalent hydrocarbon group, including a C₁-C₃₀ alkyl group, a C₂-C₃₀ alkenyl group, a C₆-C₃₀ aryl group, a C₃-C₃₀ cycloalkyl group and groups formed by replacing one or more of the carbon-bonded hydrogen atoms in those groups with halogen atom, epoxy, methacyloxy, carboxyl, carboxylate radical, sulfhydryl, polyoxyalkylene, mercapto, hydroxyl, cyano group, amino group or other substituent, such as chloromethyl, 3,3,3-trifluoropropyl, cyanomethyl, γ-aminopropyl and N-(β-aminoethyl)-γ-aminopropyl. Examples of alkyl groups are methyl, ethyl, propyl, isopropyl, butyl, tert-butyl, octyl, lauryl and stearyl groups. Examples of alkenyl groups are vinyl, allyl and propenyl. Examples of aryl groups are phenyl, tolyl, naphthyl, benzyl, phenylethyl groups, 2-phenylpropyl and xylyl. Examples of cycloalkyl groups are cyclopentyl, cyclohexyl and cyclooctyl.

The monovalent hydrocarbon group for R² group includes alkyl and alkenyl groups. Examples of alkyl groups are methyl, ethyl, propyl, isopropyl and butyl. Examples of alkenyl groups are vinyl, allyl and propenyl.

Examples of the trialkoxysilane may be methyltrialkoxysilane, methyltrimethoxysilane, methyltriethoxysilane, methyltripropoxysilane and methyltributoxysilane.

Polyalkylsilsesquioxane powder is fine polymer particles of cured polyalkylsilsesquioxane that may be in any form, such as spherical, columnar, plate and needle forms. The particle shapes can vary as necessary for the end application intended. In one embodiment, the powder particles are substantially spherical. In another embodiment, the aspect ratio, i.e., the ratio of the longer axis to the shorter axis, does not exceed about 1.2. In another embodiment, the aspect ratio does not exceed about 1.1. In one embodiment, the polyalkylsilsequioxane powder has greater than or equal to 80 percent particles having a particle diameter within the range of ±30 percent of the average particle size. In one embodiment, the average particle size is greater than or equal to about 0.05 μm and less than or equal to 100 μm. In one embodiment, the average particle size is in a range from about 0.1 μm to about 20 μm.

In one embodiment, the average particle size of the polyalkylsilsesquioxane powder emulsion is greater than or equal to about 0.05 μm. In another embodiment, the average particle size of the powder emulsion is from about 0.05 μm to about 100 μm. The average particle size includes ranges having an upper limit of about 100 μm, about 50 μm, about 20 μm and about 10 μm. The average particle size includes ranges having a lower limit of about 0.05 μm, about 0.1 μm, about 1 μm and about 2 μm. The lower limit amounts and the upper limit amounts are independently combinable to create ranges usable in the practice of the present invention. In one embodiment, the average particle size of the powder emulsion is about 20 μm. Particle sizes may be measured by means of a laser light scattering technique, using a Coulter LS230 Particle Size Analyzer.

The amount of the polyalkylsilsesquioxane powder to be formulated in the emulsion can vary depending upon the type of the emulsion desired. In one embodiment, the polyalkylsilsesquioxane powder is present in an amount from about 0.5 to about 60 percent by weight of the emulsion. The amount of polyalkylsilsesquioxane powder includes ranges having an upper limit of about 60 percent by weight, about 50 percent by weight, about 40 percent by weight, about 20 percent by weight, about 10 percent by weight and about 5 percent by weight of the emulsion. The polyalkylsilsesquioxane powder includes ranges having a lower limit of about 0.5 percent by weight, about 1 percent by weight, about 5 percent by weight, about 10 percent by weight, about 20 percent by weight and about 50 percent by weight of the emulsion. The lower limit amounts and the upper limit amounts are independently combinable to create ranges usable in the practice of the present invention.

In one embodiment, the amount of polyalkylsilsesquioxane powder in the skin care composition is from about 0.1 to about 50 percent by weight based on the weight of the composition. The amount of polyalkylsilsesquioxane powder includes ranges having an upper limit of about 50 percent by weight, about 30 percent by weight, about 20 percent by weight, about 10 percent by weight and about 5 percent by weight. The polyalkylsilsesquioxane powder includes ranges having a lower limit of about 0.1 percent by weight, about 0.2 percent by weight, about 1 percent by weight, about 2 percent by weight, about 5 percent by weight and about 10 percent by weight based on the weight of the composition. The lower limit amounts and the upper limit amounts are independently combinable to create ranges usable in the practice of the present invention.

Emulsifiers that may be used in the emulsion can be standard emulsifiers. Suitable emulsifiers are well known in the art and include nonionic, anionic, amphoteric, zwitterionic and cationic surfactants. Examples of anionic surfactants that may be used as emulsifiers are ethoxylated alkyl sulphates, alkyl ethoxy carboxylates, alkyl glyceryl ether sulphonates, acyl sarcosinates, alkyl ethoxysulphosuccinates, alpha-sulphonated fatty acids, their salts and/or esters, ethoxylated alkyl phosphate esters, ethoxylated alkyl glyceryl ether sulfonates, paraffin sulfonates and alkoxy amide sulfonates, alkyl sulphates, alkyl ether sulphates, alkaryl sulphonates, alkanoyl isethionates, alkyl succinates, alkyl sulphosuccinates, N-alkyl sarcosinates, alkyl ether phosphates, alkyl ether carboxylates, alpha-olefin sulphonates, salts of alkyl sulfates, salts of alkylbenzene sulfonates, salts of dialkyl sulfosuccinates, salts of alkyl phosphoric acids, salts of polyoxyethylene alkyl ether sulfates, glycerin monostearate, alkyl amine oxides, sorbitan monopalmitate, polyoxyethylene cetyl ether, polyoxyethylene stearic acid ester, polyoxyethylene sorbitan monolaurate and mixtures thereof. The alkyl and acyl groups generally contain from 6 to 22 carbon atoms and may be unsaturated.

Typical nonionic surfactants for use as emulsifiers include alkyl glucosides, alkyl polyglycosides, polyhydroxy fatty acid amides, alkoxylated sugar esters and polyesters, fatty acid amides, condensation products of alkylene oxides and fatty acids, such as alkylene oxide esters of fatty acids and alkylene oxide diesters of fatty acids, condensation products of alkylene oxides and fatty alcohols, such as PEG 40 hydrogenated castor oil, PEG stearate, laureth, PEG lauth ester, PEG stearate ether, steareth 2, isoceteth-20 and oleth-20, condensation products of alkylene oxides and fatty acids and fatty alcohol, wherein the polyalkylene oxide portion is esterified on one end with a fatty acid and etherified on the other end with a fatty alcohol, polyoxyethylene alkyl ethers, polyoxyethylene alkylphenyl ethers, polyoxyethylene fatty acid esters, sorbitan fatty acid esters, polyoxyethylene sorbitan fatty acid esters, glycerine fatty acid esters and saccharose fatty acid esters and Polysorbate.

Examples of amphoteric surfactants include cocoamphocarboxypropionate, cocoamphocarboxy propionic acid, cocoamphoacetate, cocoamphodiacetate, sodium lauroamphoacetate, N-acylamidopropyl-N,N-dimethylammonio betaines and N-acylamidopropyl-N,N′-dimethyl-N′-2-hydroxypropylammonio betaines.

Examples of zwitterionic surfacts include alkyl betaines and amido betaines, sultaines, alkyl glycinates, alkyl carboxyglycinates, alkyl amphopropionates, alkylamphoglycintaes, alkyl amidopropyl hydroxysulfaines, acyl taurates, acyl glutamates and examples of cationic surfactants include ammonium halide compounds, including those having hydrophilic substituents. Examples of ammonium halide compounds are long-chain alkyl trimethyl ammonium chloride, long-chain alkyl benzyl dimethyl ammonium chloride, alkylamine hydrochlorides, alkylamine acetates and di(long-chain alkyl) dimethyl ammonium bromide. Alkyl and acyl groups have from 8 to 19 carbon atoms.

In one embodiment, the emulsifier is a nonionic surfactant.

The amount of the emulsifier that may be added to the emulsion can vary depending upon the type of the emulsion desired and is not limited. In one embodiment, the emulsifier is present in an amount of from about 2 to about 50 percent by weight based on the total weight of the emulsion. The amount of emulsifier includes a range having an upper limit of about 50 percent by weight, about 30 percent by weight, about 12 percent by weight, about 10 percent by weight and about 5 percent by weight based on the weight of the total emulsion. The amount of emulsifier includes ranges having a lower limit of about 2 percent by weight, about 3 percent by weight, about 5 percent by weight and about 10 percent by weight based on the total weight of the emulsion. In another embodiment, the amount of emulsifier in the emulsion is less than 15 percent by weight based on the weight of the emulsion. The lower limit amounts and the upper limit amounts are independently combinable to create ranges usable in the practice of the present invention.

In another embodiment, the emulsifier is present from about 0.1 to about 50 percent by weight based on the total weight of the composition. The amount of emulsifier includes a range having an upper limit of about 50 percent by weight, about 40 percent by weight, about 30 percent by weight, about 20 percent by weight, about 15 percent by weight, about 13 percent by weight, about 10 percent by weight and about 5 percent by weight based on the total weight of the composition. The amount of emulsifier includes ranges having a lower limit of about 0.1 percent by weight, about 2 percent by weight, about 3 percent by weight, about 5 percent by weight and about 10 percent by weight of the total weight of the composition. In another embodiment, the amount of emulsifier is less than about 15 percent by weight based on the weight of the composition. The lower limit amounts and the upper limit amounts are independently combinable to create ranges usable in the practice of the present invention.

In one embodiment, water is present in an amount of greater than about 6 percent by weight of the emulsion. In another embodiment, the water is present in an amount of from greater than about 6 percent by weight to about 99 percent by weight and in another embodiment, the water is from about 20 percent by weight to about 99 percent by weight. The water includes ranges having an upper limit of about 99 percent by weight, about 95 percent by weight, about 90 percent by weight, about 75 percent by weight, about 50 percent by weight, about 40 percent by weight and about 30 percent by weight of the emulsion. The water includes ranges having a lower limit of about 20 percent by weight, about 25 percent by weight, about 30 percent by weight, about 40 percent by weight, about 50 percent by weight and about 60 percent by weight of the emulsion. The lower limit amounts and the upper limit amounts are independently combinable to create ranges usable in the practice of the present invention.

In one embodiment, the water in the water-based skin care cosmetic composition is present in an amount of from about 0.2 to about 99.5 percent by weight of the total composition. The amount of the water includes ranges having an upper limit of about 99.5 percent by weight, about 99 percent by weight, about 95 percent by weight, about 80 percent by weight, about 70 percent by weight and about 40 percent by weight. The amount of the water includes ranges having a lower limit of about 0.2 percent by weight, about 10 percent by weight, about 20 percent by weight, about 50 percent by weight and about 70 percent by weight of the total composition. In one embodiment, the water is present in an amount of greater than about 6 percent by weight based on the total weight of the composition. The lower limit amounts and the upper limit amounts are independently combinable to create ranges usable in the practice of the present invention.

The emulsion to be added to the skin care composition varies depending on the type of cosmetic desired and is not limited. In one embodiment, the emulsion is present in the composition in an amount of from about 0.2 to about 99.8 percent by weight of the composition. The amount of emulsion includes ranges having an upper limit of about 99.8 percent by weight, about 90 percent by weight, about 80 percent by weight, about 50 percent by weight, about 40 percent by weight, about 30 percent by weight, about 20 percent by weight, about 10 percent by weight and about 5 percent by weight of the composition. The amount of emulsion includes ranges having a lower limit of about 0.2 percent by weight, about 0.5 percent by weight, about 1 percent by weight, about 5 percent by weight, about 10 percent by weight, about 20 percent by weight and about 30 percent by weight based on the total weight of the composition. The lower limit amounts and the upper limit amounts are independently combinable to create ranges usable in the practice of the present invention.

In another embodiment, a water-based skin care cosmetic composition comprises a polyalkylsilsesquioxane powder emulsion having an average particle diameter in the range of about 0.05 to about 100 μm, an emulsifier and water, wherein said emulsifier is less than 15 percent by weight and the water is greater than 6 percent by weight, wherein said percent by weight is based on the total weight of the composition.

In another embodiment, the skin care composition may optionally comprise a silicone to provide the composition with an improved long lasting silkness, softness and emollient touch.

The silicone may be a crosslinked silicone copolymer or a linear or branched polyorganosiloxane. In one embodiment, the silicone has a viscosity in the range of from about 3 centistokes to about 30,000,000 centipoise. The viscosity of the silicone includes ranges having an upper limit of about 30,000,000 centipoise, about 20,000,000 centipoise, about 10,000,000 centipoise, about 1,000,000 centipoise and about 10,000 centistokes. The viscosity of the silicone includes ranges having a lower limit of about 3 centistokes, about 5 centistokes, about 10 centistokes, about 20 centistokes, about 350 centistokes, about 820 centistokes and about 10,000 centistokes. The lower limit amounts and the upper limit amounts are independently combinable to create ranges usable in the practice of the present invention. The viscosity is measured by Brookfield DV-II or HAAKE RheoStress 600 at 25° C.

In one embodiment, the silicone may be a blend of a low viscosity silicone and a high viscosity silicone. In one embodiment, the low viscosity silicone may be in a range of from about 3 centistokes to about 10,000 centistokes. The viscosity of the low viscosity silicone includes ranges having an upper limit of about 10 centistokes, about 20 centistokes, about 350 centistokes, about 820 centistokes and about 10,000 centistokes. The viscosity of the low viscosity silicone includes ranges having a lower limit of about 3 centistokes, about 5 centistokes, about 10 centistokes, about 20 centistokes, about 350 centistokes, and about 800 centistokes. The lower limit amounts and the upper limit amounts are independently combinable to create ranges usable in the practice of the present invention.

In another embodiment, the high viscosity silicone may be in a range of from about 1,000,000 centipoise to about 30,000,000 centipoise. The viscosity of the high viscosity silicone includes ranges having an upper limit of 10,000,000 centipoise, about 20,000,000 centipoise and about 30,000,000 centipoise. The viscosity of the high viscosity silicone includes ranges having a lower limit of about 1,000,000 centipoise, about 10,000,000 centipoise and about 20,000,000 centipoise. The lower limit amounts and the upper limit amounts are independently combinable to create ranges usable in the practice of the present invention.

In another embodiment, the polyorganosiloxane has the formula:

MD_{q}TₛM

wherein:

### M is R³₃SiO_{1/2};

### D is R⁴₂SiO_{2/2};

### T is R⁵SiO_{3/2}

R³, R⁴ and R⁵ are each independently alkyl, aryl or aralkyl containing from one to sixty carbon atoms, including one to 30 carbon atoms and one to 10 carbon atoms; q and s are each independently integers from 0 to 300. In another embodiment q and s are each independently integers from 0 to 50 and in another embodiment, q and s are each independently integers from 0 to 20.

In one embodiment, the silicone is a linear or branched polydimethylsiloxane.

In one embodiment, the silicone is a crosslinked silicone copolymer. Some examples of organopolysiloxanes are C₃₀-C₄₅ alkyl cetearyl dimethicone crosspolymer with cyclopentasiloxane as carrier (sold as GE brand name Velvesil® 125), cetearyl dimethicone crosspolymer with dimethicone as carrier (sold as GE brand name Velvesil® DM) and vinyl dimethicone crosspolymer with cyclopentasiloxane and dimethicone as carrier (sold as GE brand name SFE839).

The amount of silicone that may be optionally added to the composition may vary depending on the type of composition desired and is not limited. In one embodiment, the silicone is present in an amount of from about 0.1 to about 50 by weight percent based on the weight of the composition. The amount of silicone includes a range having an upper limit of about 50 percent by weight, about 30 percent by weight, about 20 percent by weight, about 10 percent by weight and about 5 percent by weight based on the weight of the composition. The amount of silicone includes a range having a lower limit of about 0.5 percent by weight, about 1 percent by weight, about 2 percent by weight, about 5 percent by weight, about 10 percent by weight, about 20 percent by weight, about 30 percent by weight and about 40 percent by weight based on the weight of the composition. The lower limit amounts and the upper limit amounts are independently combinable to create ranges usable in the practice of the present invention.

The silicone may be blended with the polyalkylsilsesquioxane powder and incorporated into the emulsion or may be blended with the polyalkylsilsesquioxane powder emulsion into the cosmetic composition.

The amount of silicone that may be optionally added to the emulsion may vary depending on the type of emulsion desired and is not limited. In one embodiment, the silicone is present in an amount of from about 0.1 to 50 weight percent based on the weight of the emulsion. The amount of silicone includes a range having an upper limit of about 50 percent by weight, about 30 percent by weight, about 25 percent by weight, about 20 percent by weight, about 10 percent by weight and about 5 percent by weight based on the weight of the emulsion. The amount of silicone includes a range having a lower limit of about 0.1 percent by weight, about 0.5 percent by weight, about 1 percent by weight, about 5 percent by weight, about 10 percent by weight, about 20 percent by weight and about 30 percent by weight based on the weight of the emulsion. The lower limit amounts and the upper limit amounts are independently combinable to create ranges usable in the practice of the present invention.

In another embodiment, the skin care composition optionally comprises a wax. The wax may be a silicone wax, a petroleum wax, plant wax or animal wax. The wax provides the composition with improved moisture retention, longer lasting sensory on the skin, increases the viscosity and provides good shelf-life stability. Some examples of waxes are bees wax, microcrystalline wax, ozokerite, ceresin and candelilla wax. A silicone wax may be a C₈ or higher alkyl-modified methicone or dimethicone and has a melting point in the range of from about 30° C. to about 90° C. In one embodiment, the silicone wax is a C₈-C₃₀ alkyl-modified methicone or dimethicone. In another embodiment, the silicone wax is a cetearyl methicone.

In one embodiment, a wax may be added to the composition in the amount of from about 0 to about 25 percent by weight of the composition, including 0.1 to about 25 percent by weight of the composition. The amount of wax includes a range having an upper limit of about 25 percent by weight, about 20 percent by weight, about 10 percent by weight and about 5 percent by weight based on the total weight of the composition. The amount of wax includes a range having a lower limit of about 0.1 percent by weight, about 0.2 percent by weight, about 1 percent by weight, about 5 percent by weight and about 10 percent by weight based on the total weight of the composition. The lower limit amounts and the upper limit amounts are independently combinable to create ranges usable in the practice of the present invention.

The wax may be blended with the polyalkylsilsesquioxane powder into the emulsion or may be blended with the polyalkylsilsesquioxane powder emulsion into the cosmetic composition.

In one embodiment, a wax may be added to the emulsion in an amount of from about 0 to about 25 percent by weight of the emulsion, including 0.2 to about 25 percent by weight of the emulsion. The amount of wax includes a range having an upper limit of about 25 percent by weight, about 20 percent by weight, about 10 percent by weight and about 5 percent by weight based on the total weight of the emulsion. The amount of wax includes a range having a lower limit of about 0.2 percent by weight, about 1 percent by weight, about 5 percent by weight and about 10 percent by weight based on the total weight of the emulsion. The lower limit amounts and the upper limit amounts are independently combinable to create ranges usable in the practice of the present invention.

In another embodiment, the skin care composition optionally comprises an emollient. The emollient may be incorporated into the polyalkylsilsesquioxane powder emulsion or separately into the skin care composition. The emollient includes oily materials such as various kinds of hydrocarbons, higher fatty acids, oils and fats, esters, higher alcohols, lower alcohols, such as octyl alcohol, ethanol, castor oil, squalane, liquid paraffin, isoparaffin, petroleum jelly, myristic acid, palmitic acid, stearic acid, oleic acid, isostearic acid, cetyl alcohol, stearyl alcohol, hexadecyl alcohol, oleyl alcohol, cetyl 2-ethylhexanoate, 2-ethylhexyl palmitate, 2-octyldodecyl myristate, 2-octyldodecyl gum esters, neopentyl glycol 2-ethylhexanoate, isooctylic acid triglyceride, 2-cotyldodecyl oleate, isopropyl myrisate, isopropyl palmitate, isostearic acid triglycieride, 2-octyldodecyl oleate, isopropyl myristate, isopropyl palmitate, isostearic acid triglyceride, coconut oil fatty acid triglyceride, olive oil, avocado oil, myristyl myrisate, mink oil, lanolin, amino acid compounds, sodium pyrrolidone carboxylic acid, D-panthernol, lactic acid, L-proline, guanidine, pyrrolidone, hydrolyzed protein and other collagen-derived proteins, aloe vera gel, acetamide MEA and lactamide MEA and ethyl acetate resins such as alkyd resins, acrylic resins and urea resins.

In one embodiment, an emollient may be added to the composition in the amount of from about 0 to about 25 percent by weight of the composition, including 0.2 to about 25 percent by weight of the composition. The amount of emollient includes a range having an upper limit of about 25 percent by weight, about 20 percent by weight, about 10 percent by weight and about 5 percent by weight based on the total weight of the composition. The amount of emollient includes a range having a lower limit of about 0.2 percent by weight, about 1 percent by weight, about 5 percent by weight and about 10 percent by weight based on the total weight of the composition. The lower limit amounts and the upper limit amounts are independently combinable to create ranges usable in the practice of the present invention.

In one embodiment, an emollient may be added to the emulsion in an amount of from about 0 to about 25 percent by weight of the emulsion, including 0.2 to about 25 percent by weight of the emulsion. The amount of emollient includes a range having an upper limit of about 25 percent by weight, about 20 percent by weight, about 10 percent by weight and about 5 percent by weight based on the total weight of the emulsion. The amount of emollient includes a range having a lower limit of about 0.2 percent by weight, about 1 percent by weight, about 5 percent by weight and about 10 percent by weight based on the total weight of the emulsion. The lower limit amounts and the upper limit amounts are independently combinable to create ranges usable in the practice of the present invention.

In another embodiment, an emulsion comprises polyalkylsilsesquioxane powder having an average particle diameter in the range of about 0.05 to about 100 μm, a silicone, an emulsifier and water, wherein said emulsifier is less than 15 percent by weight and the water is greater than about 6 percent by weight, wherein the percent by weight is based on the total weight of the emulsion.

Additives conventionally used for cosmetics may optionally be selected and added to the cosmetic composition according to the type of the cosmetic composition desired. The types and amounts of additives selected will vary with the type of cosmetic composition and are not limited. Some examples of additives include plasticizers such as camphor and acetyl tributyl citrate and other ingredients such as ultraviolet absorbers, antioxidants, antiseptics, humectants, perfumes, alcohols, pigments, including pearlescent pigments such as, bismuth oxychloride and titanium dioxide coated mica, colorants, fragrances, biocides, preservatives, antioxidants, anti-microbial agents, anti-fungal agents, exfoliants, hormones, enzymes, medicinal compounds, vitamins, salts, electrolytes, alcohols, polyols, botanical extracts, film formers, thickening agents such as an acrylate crosspolymer, acrylate/C₁₀-C₃₀ alkyl acrylate crosspolymer, fumed silica or hydrated silica, particulate fillers, such as talc, kaolin, starch, modified starch, mica, nylon and clays, such as, bentonite and organo-modified clays.

The amount of additives that may be added to the composition may vary depending on the type of composition desired and is not limited. In one embodiment, the additives are present in an amount of from about 0 to about 60 weight percent based on the weight of the emulsion. The amount of additives includes a range having an upper limit of about 60 percent by weight, about 50 percent by weight, about 25 percent by weight, about 10 percent by weight and about 5 percent by weight based on the weight of the composition. The amount of additives includes a range having a lower limit of about 0 percent by weight, about 0.1 percent by weight, about 0.2 percent by weight, about 0.5 percent by weight, about 1 percent by weight, about 5 percent by weight, about 10 percent by weight, about 20 percent by weight, about 30 percent by weight and about 50 percent by weight based on the total weight of the composition. The lower limit amounts and the upper limit amounts are independently combinable to create ranges usable in the practice of the present invention.

The additives can be blended into the composition by blending with the polyalkylsilsesquioxane powder emulsion or into the skin care composition by conventional means.

The water-based skin care compositions may be used in facial masks, wipes, make-up remover, oil-in-water creams and lotions and rinse-off skin care compositions, such as body cleansers, skin cleansers, gel or lotion-type facial masks, shower products and hand cleansers.

In one embodiment of the invention, the composition is applied to a facial mask or wipe. The facial masks and wipes may be tissues, wipes, towels, towelettes and the like. The materials may be biodegradable. Facial masks and wipes typically are manufactured by impregnating sheets made of non-woven fabric with a suitable gel, lotion or solution.

In one embodiment, a facial mask comprises a skin care composition comprising from about 0.5 to about 40 parts by weight of polyalkylsilsesquioxane powder emulsion and from about 20 to about 99 parts by weight water. The parts by weight are based on the total weight of the cosmetic composition.

In one embodiment, a wipe comprises a skin care composition comprising about 0.5 to about 40 parts by weight of polyalkylsilsesquioxane powder emulsion and from about 20 to about 99 parts by weight water. The parts by weight are based on the total weight of the cosmetic composition.

In one embodiment, a body wash comprises a skin care composition comprising about 0.5 to about 40 parts by weight of polyalkylsilsesquioxane powder emulsion and from about 20 to about 99 parts by weight water. The parts by weight are based on the total weight of the cosmetic composition.

### EXAMPLES

### Example 1

An emulsion was prepared by blending 15 parts by weight of a polydimethylsiloxane (GE Brand name XF49-813) and 6 parts by weight of a dimethicone (GE brand name SF96-10) having a viscosity of 10 centistokes as measured by Brookfield DV-II+ at 25° C. The blended polydimethylsiloxane having a viscosity of about 819 centistokes was added to a flask. 25 parts by weight of polymethylsilsesquioxane powder (Tospearl® 145) with an average particle size of about 4.5 μm was added to the polydimethylsiloxane mixture and stirred. 8 parts by weight of isohexadecane was added to the mixture and stirred. The mixture was heated to 60° C. 3 parts by weight of cetearyl methicone, 5 parts by weight of PEG stearate 40, 3.0 parts by weight of PEG-4 Lauth ester and 2.0 parts by weight of PEG-23 Lauth ester were added to the mixture and stirred. 33 parts by weight of water was slowly added to the mixture and mixed while heating. An emulsion formed and was allowed to cool to room temperature. The emulsion had an average particle size of about 2.0 to 5.0 μm, measured by a laser light scattering technique, using a Coulter LS230 Particle Size Analyzer.

### Example 2

A facial mask or body lotion was prepared by mixing together 2.0 parts by weight 1,3-butylene glycol, 4.0 parts by weight glycerin, 0.15 parts by weight Carbopol® 941 (Carbopol® 941 is an acrylate crosspolymer), 5.0 parts by weight of the polymethylsilsesquioxane powder emulsion from Example 1, 2-3 drops of a preservative and 89.7 parts by weight water. The mixture is neutralized with 0.15 parts by weight triethanolamine. The mixture is blended together to form a gel. The gel can be applied directly to the face as a facial mask, to the body as a body lotion, or applied to a non-woven sheet by soaking the sheet in the gel as a different type of facial mask.

### Example 3

Wipes were prepared by mixing together 1.2 parts by weight polysorbate 20, 1.0 part by weight PEG-75 Lanolin, 3.5 parts by weight PEG-40 hydrogenated castor oil, 5.0 parts by weight propylene glycol, 7.5 parts by weight of a polymethylsilsesquioxane powder emulsion, 2-3 drops of perfume and 81.8 parts by weight water. The mixture is blended together to form a lotion, which is applied to a towelette.

### Example 4

A body wash was prepared by mixing together 70.8 parts by weight water, 1.8 parts by weight cocamidopropyl betaine, 10.5 parts by weight ammonium laureth sulfate and 0.2 parts by weight Carbopol® Ultrez 20 (an acrylate/C₁₀-C₃₀ alkyl acrylate crosspolymer.). The mixture was neutralized with 0.20 parts by weight triethanolamine. To the mixture, was then added 1 drop fragrance, 2 drops preservative, 9.0 parts by weight NaCl and 7.5 parts by weight of the polymethylsilsesquioxane powder emulsion prepared in Example 1. The mixture was blended together to form a gel.

### Example 5

A body wash was prepared by mixing together 72.5 parts by weight water, 1.8 part by weight cocamidopropyl betaine, 15.4 parts by weight ammonium laureth sulfate and 0.4 parts by weight Carbopol® Ultrez 20 (Carbopol® Ultrez 20 is an acrylate/C₁₀-C₃₀ alkyl acrylate crosspolymer.). The mixture was neutralized with 0.40 parts by weight triethanolamine. 2 drops of a preservative, 2.0 parts by weight NaCl and 7.5 parts by weight of a polymethylsilsesquioxane powder emulsion were added to the mixture. The mixture was blended together to form a gel.

### Example 6

Facial mask/body lotion

| Ingredients | A | B |
|---|---|---|
| 1,3 Butylene Glycol | 2 | 2 |
| Glycerin | 4 | 4 |
| Carbopol ® 941¹ | 0.15 | 0.15 |
| Water | Add to 100 | Add to 100 |
| Triethanolamine | 0.15 | 0.15 |
| Polymethylsilsesquioxane | — | 5 |
| powder emulsion from | | |
| Example 1 | | |
| Preservative | a.q | a.q |

¹Carbopol ® 941 is an acrylate crosspolymer

Facial mask B was prepared in accordance with Example 2. Facial mask A was prepared in accordance with Example 2 except without the polymethylsilsesquioxane powder emulsion. Polymethylsilsesquioxane powder could also not be added to Facial Mask A, as the powder was not compatible with the mixture. The property results are shown in Table 1. Facial mask B has improved smoothness and softness, color and compatibility.

### Example 7

Wipes

| Ingredients | C % | D % |
|---|---|---|
| Polysorbate 20 | 1.25 | 1.25 |
| PEG-75 Lanolin | 1 | 1 |
| PEG-40 Hydrogenated Castor | 3.5 | 3.5 |
| Oil | | |
| Propylene Glycol | 5 | 5 |
| Polymethylsilsesquioxane | — | 7.5 |
| powder emulsion from Ex. 1 | | |
| Water | Add to 100 | Add to 100 |
| Preservative | a.q. | a.q. |
| Perfume | a.q. | a.q. |

Wipes D were prepared in accordance with Example 3. Wipes C were prepared in accordance with Example 3, except without the polymethylsilsesquioxane powder emulsion. Polymethylsilsesquioxane powder could also not be added to the Wipes C, as the powder was not compatible with the mixture. The property results are shown in Table 1. Wipes D have improved smoothness and softness, color and compatibility.

### Example 8

Body wash

| Ingredients | E % | F % |
|---|---|---|
| Cocamidopropyl betaine | 1.8 | 1.8 |
| Ammonium laureth sulfate | 10 | 10 |
| Carbopol ® Ultrez 20¹ | 0.2 | 0.2 |
| Triethanolamine | 0.20 | 0.20 |
| Polymethylsilsesquioxane | — | 7.5 |
| powder emulsion Ex. 1 | | |
| Polymethylsilsesquixoane | 1.9 | — |
| powder² | | |
| Water | Add to 100 | Add to 100 |
| Fragrance | q.s. | q.s. |
| NaCl | 6.0 | 9.0³ |

¹Carbopol ® Ultrez 20 is an acrylate/C₁₀–C₃₀ alkyl acrylate crosspolymer.
²Polymethylsilsesquioxane powder is Tospearl ® 145 with an average particle size of 4.5 μm.
³When the powder emulsion is added, the viscosity decreases, so an increased level of NaCl is needed to maintain the viscosity of the water phase.

Body wash F was prepared in accordance with Example 4. Body wash E was prepared in accordance with Example 4 except the polymethylsilsesquioxane powder was used in place of the polymethylsilsesquioxane powder emulsion. The polymethylsilsesquioxane powder initially agglomerated on top of the mixture, but dispersed after several minutes of mixing. The property results are shown in Table 1. Body Wash F has improved smoothness and softness.

### Example 9

Body wash

| Ingredients | G % | H % |
|---|---|---|
| Cocamidopropyl betaine | 1.8 | 1.8 |
| Ammonium laureth sulfate | 15.4 | 15.4 |
| Carbopol ® Ultrez 20¹ | 0.4 | 0.4 |
| Triethanolamine | 0.4 | 0.4 |
| Polymethylsilsesquioxane | — | 7.5 |
| powder emulsion Ex. 1 | | |
| Polymethylsilsesquixoane | 1.9 | — |
| powder² | | |
| Water | Add to 100 | Add to 100 |
| Fragrance | q.s. | q.s. |
| NaCl | 1.5 | 2.0³ |

¹Carbopol ® Ultrez 20 is an acrylate/C₁₀–C₃₀ alkyl acrylate crosspolymer.
²Polymethylsilsesquioxane powder is Tospearl ® 145 with an average particle size of 4.5 μm.
³When the powder emulsion is added, the viscosity decreases, so an increased level of NaCl is needed to maintain the viscosity of the water phase.

Body wash H was prepared in accordance with Example 4. Body wash G was prepared in accordance with Example 4 except the polymethylsilsesquioxane powder was used in place of the polymethylsilsesquioxane powder emulsion. The polymethylsilsesquioxane powder initially agglomerated on top of the mixture, but dispersed after several minutes of mixing. The property results are shown in Table 1. Body Wash H has improved smoothness and softness.

### Example 10

Suncare lotion/cream

| | I | J |
|---|---|---|
| Part A | | |
| POE(2)stearyl ether | 2.5 | 2.5 |
| POE(21)stearyl ether | 2 | 2 |
| Cyclopentasiloxane (SF 1202) | 3 | 3 |
| Cyclopentasiloxane (and) C_{30–45} Alkyl | 5 | 5 |
| Cetearyl Dimethicone Crosspolymer | | |
| (Velvesil125) | | |
| Caprylyl Methicone (Silsoft 034)) | 3 | 3 |
| Cyclopentasiloxane and | 0.5 | 0.5 |
| Trimethylsiloxysilicate (SS4230) | | |
| Tocopherol acetate USP | 0.3 | 0.3 |
| Benzophenone-3 | 2 | 2 |
| Octyl MethoxyCinnamate | 4 | 4 |
| Butylmethoxydibenzoylmethane | 0.3 | 0.3 |
| Part B | | |
| Water deionized | Add to 100 | Add to 100 |
| Glycerin | 5 | 5 |
| Niacinamide | 2 | 2 |
| Panthenol | 1 | 1 |
| Arbutin | 0.05 | 0.05 |
| Na₄EDTA | 0.08 | 0.08 |
| Sodium Ascorbyl phosphate | 0.04 | 0.04 |
| Carbopol 941 | 0.16 | 0.16 |
| Triethanolamine | 0.16 | 0.16 |
| Part C | | |
| Preservative | q.s. | q.s. |
| Fragrance | q.s. | q.s. |
| Part D | | |
| Polymethylsilsesquioxane powders | | 10 |
| Emulsion from Ex. 1 | | |

Ingredients of Part A were combined and heated to melt. Ingredients of Part B were combined and heated to 75° C. The Part B mixture was slowly added to the Part A mixture and the mixture of Parts A and B were homogenized. The mixture from Parts A and B was cooled to 30° C. with moderate mixing. The fragrance and preservative were added. Part D was added to the overall mixture and stirred for 10 minutes to prepare the product.

A skin lightening lotion with UVA/UVB Protection was prepared in accordance with Sample J. Sample I was prepared without the polymethylsilsesquioxane powder emulsion and without polymethylsilsesquioxane powder, as the powder was not compatible with the mixture when added after the emulsion was prepared. The property results are shown in Table 1. Skin lightening lotion with UVA/UVB Protection Sample J has improved smoothness and softness.

### Example 11

Oil make up-remover

| Ingredients | K % | L % |
|---|---|---|
| Polymethylsilsesquioxane | — | 93 |
| powders Emulsion (Ex. 1) | | |
| water | Add to 100 | Add to 100 |

Make up-remover Samples K and L were prepared and the property results are shown in Table 1. Make up-remover L has greatly improved cleaning, smoothness and softness. Sample K was prepared without the polymethylsilsesquioxane powder emulsion and without polymethylsilsesquioxane powder, as the powder was not compatible with the water.

TABLE 1

| | A | B | | | | | | | | | | |
|---|---|---|---|---|---|---|---|---|---|---|---|---|
| | Facial | Facial | | | | | | | I | J | | |
| | mask/ | mask/ | | | E | F | G | H | Skin | Skin | K | L |
| | body | body | C | D | Body | Body | Body | Body | lightening | lightening | Make-up | make up |
| | lotion | lotion | Wipes | Wipes | wash | wash | wash | wash | lotion | lotion | remover | remover |
| Smooth | 2.2 | 3.9 | 2.5 | 4.0 | 3.0 | 4.0 | 3.0 | 4.0 | 2.2 | 3.5 | 0.5 | 3.2 |
| application | | | | | | | | | | | | |
| Softness | 2.0 | 3.9 | 2.3 | 4.2 | 3.0 | 4.2 | 2.2 | 3.0 | 3.0 | 3.6 | 0.2 | 3.5 |
| Absorbance | 3.0 | 3.9 | 1.0 | 1.0 | N/A | N/A | N/A | N/A | 2.0 | 3.1 | 0.5 | 4 |
| Whitening | 0.3 | 3.4 | 0.1 | 0.6 | 2.0 | 2.5 | 3.0 | 3.5 | 2.0 | 2.5 | 0.1 | 3.5 |
| Anti-wrinkle | 0.2 | 4.0 | 0.1 | 1.0 | 2.0 | 2.5 | 3.0 | 3.5 | 1.0 | 2.3 | 0.2 | 2.2 |
| Anti-tacky | 1.0 | 3.5 | 0.2 | 3.0 | N/A | N/A | N/A | N/A | 1.0 | 3.0 | | |
| Moisture | 2.0 | 4.2 | 2.0 | 4.0 | 2.5 | 4.0 | 2.0 | 3.5 | 2.0 | 2.5 | 0.3 | 3 |
| Oil Make-up Cleaning | N/A | N/A | N/A | N/A | N/A | N/A | N/A | N/A | N/A | N/A | 0.5 | 4.5 |

The performance of the examples above is evaluated by panelists with 1-5 score where 5 is excellent and 1 is poor.

While typical embodiments have been set forth for the purpose of illustration, the foregoing descriptions should not be deemed to be a limitation on the scope herein. Accordingly, various modifications, adaptations and alternatives may occur to one skilled in the art without departing from the spirit and scope herein.

Having described the invention, that which is claimed is:

1. A process for making a water-based skin care composition comprising blending a polyalkylsilsesquioxane powder emulsion and water.

2. The process of claim 1 wherein the composition is an oil-in-water emulsion or an aqueous solution.

3. The process of claim 1 wherein the polyalkylsilsesquioxane powder emulsion comprises polyalkylsilsesquioxane powder and water.

4. The process of claim 1 wherein polyalkylsilsesquioxane is represented by the average unit formula of R¹SiO_{1.5}, wherein R¹ is a substituted or unsubstituted monovalent hydrocarbon group.

5. The process of claim 1 wherein polyalkylsilsesquioxane is polymethylsilsesquioxane, polyethylsilsesquioxane or polyphenylsilsesquioxane.

6. The process of claim 1 wherein the average particle size of the polyalkylsilsesquioxane powder emulsion is greater than or equal to about 0.05 μm.

7. The process of claim 1, wherein the process further comprises adding an emulsifier.

8. The process of claim 1 wherein the water is present in an amount of from about 0.2 to about 99.5 percent by weight based on the total weight of the composition.

9. The process of claim 1 wherein the polyalkylsilsesquioxane powder emulsion is present in the composition in an amount of from about 0.2 to about 99.8 percent by weight based on the total weight of the composition.

10. The process of claim 1 wherein the process further comprises adding a silicone.

11. The process of claim 1 wherein the process further comprises adding a wax.

12. The process of claim 1 wherein the process further comprises adding an emollient.

13. A water-based skin care cosmetic composition comprising a polyalkylsilsesquioxane powder emulsion having an average particle diameter in the range of about 0.05 μm to about 100 μm, an emulsifier and water, wherein said emulsifier is less than 15 percent by weight and said water is greater than about 6 percent by weight, wherein said percent by weight is based on the total weight of the composition.

14. The composition of claim 13, wherein the composition is an oil-in-water emulsion or an aqueous solution.

15. The composition of claim 13 wherein the polyalkylsilsesquioxane powder emulsion comprises polyalkylsilsesquioxane powder and water.

16. The composition of claim 13 wherein polyalkylsilsesquioxane is represented by the average unit formula of R¹SiO_{1.5}, wherein R¹ is a substituted or unsubstituted monovalent hydrocarbon group.

17. The composition of claim 16 wherein R¹ is a C₁-C₃₀ alkyl group, a C₂-C₃₀ alkenyl group, a C₆-C₃₀ aryl group or a C₃-C₃₀ cycloalkyl group.

18. The composition of claim 13 wherein polyalkylsilsesquioxane is polymethylsilsesquioxane, polyethylsilsesquioxane or polyphenylsilsesquioxane.

19. The composition of claim 13, wherein the average particle size of the polyalkylsilsesquioxane powder emulsion is from about 0.05 to about 50 μm.

20. The composition of claim 13, wherein the emulsifier is selected from the group consisting of nonionic surfactants, anionic surfactants, amphoteric surfactants, zwitterionic surfactants and cationic surfactants.

21. The composition of claim 13, wherein the emulsifier is present in an amount from about 2 to about 13 percent by weight based on the total weight of the composition.

22. The composition of claim 13 wherein the water is present in an amount from about 10 to about 99.5 percent by weight based on the total weight of the composition.

23. The composition of claim 13 wherein the polyalkylsilsesquioxane powder emulsion is present in the composition in an amount of from about 0.2 to about 99.8 percent by weight based on the total weight of the composition.

24. The composition of claim 13 further comprising a silicone.

25. The composition of claim 13 further comprising a wax.

26. The composition of claim 13 further comprises an emollient.

27. An emulsion comprising polyalkylsilsesquioxane powder having an average particle diameter in the range of about 0.05 to about 100 μm, a silicone, an emulsifier and water, wherein said emulsifer is less than about 15 percent by weight and the water phase is greater than about 6 percent by weight, wherein the percent by weight is based on the total weight of the emulsion.

28. The emulsion of claim 27 wherein polyalkylsilsesquioxane is represented by the average unit formula of R¹SiO_{1.5}, wherein R¹ is a substituted or unsubstituted monovalent hydrocarbon group.

29. The emulsion of claim 27 wherein polyalkylsilsesquioxane is polymethylsilsesquioxane, polyethylsilsesquioxane or polyphenylsilsesquioxane.

30. The emulsion of claim 27, wherein the average particle size of the polyalkylsilsesquioxane powder emulsion is from about 0.05 to about 50 μm.

31. The emulsion of claim 27 wherein the polyalkylsilsesquioxane powder is present from about 0.5 to about 60 percent by weight based on the total weight of the emulsion.

32. The emulsion of claim 27, wherein the emulsifier is selected from the group consisting of nonionic surfactants, anionic surfactants, amphoteric surfactants, zwitterionic surfactants and cationic surfactants.

33. The emulsion of claim 27, wherein the emulsifier is present in an amount from about 2 to about 12 percent by weight based on the total weight of the emulsion.

34. The emulsion of claim 27, wherein the water phase is present in an amount of from about 20 to about 99 percent by weight of the emulsion.

35. The emulsion of claim 27 wherein the silicone is a crosslinked silicone copolymer or a linear or branched polyorganosiloxane.

36. The emulsion of claim 27 wherein the silicone is present in an amount of from about 0.1 to about 50 weight percent based on the total weight of the emulsion.

37. The emulsion of claim 27 further comprising a wax.

38. The emulsion of claim 27 further comprises an emollient.

39. A facial mask comprising a skin care composition comprising from about 0.5 to about 40 percent by weight of polyalkylsilsesquioxane powder emulsion and from about 20 to about 99 parts by weight water, wherein the parts by weight are based on the total weight of the cosmetic composition.

41. A wipe comprising a skin care composition comprising from about 0.5 to about 40 parts by weight of polyalkylsilsesquioxane powder emulsion and from about 20 to about 99 parts by weight water, wherein the parts by weight are based on the total weight of the cosmetic composition.

42. A body wash comprising a skin care composition comprising from about 0.5 to about 40 parts by weight of polyalkylsilsesquioxane powder emulsion and from about 20 to about 99 parts by weight water, wherein the parts by weight are based on the total weight of the cosmetic composition.

43. A skin care composition prepared by the method in claim 1.
